(19) United States
(12) Patent Application Publication
(10) Pub. No.: US 2014/0135644 A1
(43) Pub. Date: May 15, 2014

(54) WEARABLE DEVICE AND MANAGING DEVICE TO MANAGE STATUS OF USER, AND METHODS THEREOF

(71) Applicant: Samsung Electronics Co., Ltd, Suwon-si (KR)

(72) Inventor: Sang-tae KIM, Suwon-si (KR)

(73) Assignee: Samsung Electronics Co., Ltd, Suwon-si (KR)

(21) Appl. No.: 14/079,773

(22) Filed: Nov. 14, 2013

(30) Foreign Application Priority Data
Nov. 15, 2012 (KR) 10-2012-0129808

(51) Int. Cl. A61B 5/0482; A61B 5/11; A61B 5/04; A61B 5/00

(52) U.S. Cl. CPC A61B 5/0482; A61B 5/6803; A61B 5/7455; A61B 5/742; A61B 5/0006; A61B 5/1112; A61B 5/746; A61B 5/04012
USPC 600/545

(57) ABSTRACT

A wearable device and a managing device to manage a status of a user, the wearable device including a brainwave measuring sensor to measure a brainwave of the user, an environment information sensor to sense surrounding environment information, a vibrator to generate vibrations, a display device to display a message, a communicator to transmit sensed values of the brainwave measuring sensor and the environment information sensor to an external device and to receive a control signal from the external device, and a controller to control operations of the vibrator and the display device based on at least one of the sensed value of the brainwave measuring sensor, the sensed value of the environment sensor, and the control signal. Therefore, safety of the user is maintained even when the use is in a dangerous situation.

### CROSS-REFERENCE TO RELATED APPLICATIONS

This application claims priority under 35 U.S.C. §119 from Korean Patent Application No. 10-2012-0129808, filed on Nov. 15, 2012, in the Korean Intellectual Property Office, the disclosure of which is incorporated herein by reference in its entirety.

### BACKGROUND OF THE INVENTION

1. Field of the Invention

The present general inventive concept generally relates to providing a wearable device and a managing device, and methods of managing a status of a user by using the wearable device and the managing device, and more particularly, to providing a wearable device that a user wears on a body of the user, a managing device to manage a status of the user according to a value sensed by the wearable device, and methods thereof.

2. Description of the Related Art

Development of human civilization requires research and work to be performed even in dangerous environments such as under water, in a jungle, in a very hot place, in a very cold place, etc. Work is frequently performed even at a scene of a fire or at a scene of a radioactive leak or toxic waste spill.

In these cases, workers are in danger of losing their consciousnesses due to poisonous gases, heat, cold, etc. Since it is difficult for a worker to identify these hazardous elements with the naked eye, the worker does not easily recognize a hazardous situation until the worker is in danger. As such, a majority of workers are unable to clearly view imminent dangers due to smoke, darkness, etc., and thus are unable to rapidly perceive and identify potentially hazardous situations. For these reasons, workers who work in manholes, at fire scenes, with nuclear reactors, etc. frequently lose their lives.

In order to prevent accidents as described above, various types of safety devices are frequently installed at work sites. However, the safety devices are not used at all times, due to lack of manpower, restrictions regarding work speed, unusual work environments, emergency situation, etc. Accordingly, a technology to check a status of a user or a surrounding environment to prevent a danger is required.

### SUMMARY OF THE INVENTION

The present general inventive concept provides a wearable device that a user wears on a body of the user to sense a status and a surrounding environment of the user and cope with a hazardous situation according to the sensed result, a managing device that uses the wearable device, and methods thereof.

Additional features and utilities of the present general inventive concept will be set forth in part in the description which follows and, in part, will be obvious from the description, or may be learned by practice of the general inventive concept.

The foregoing and/or other features and utilities of the present general inventive concept are achieved by providing a wearable device that a user wears on a body of the user, including a brainwave measuring sensor to measure a brainwave of the user, an environment information sensor to sense surrounding environment information, a vibrator to generate vibrations, a display device to display a message, a communicator to transmit sensed values of the brainwave measuring sensor and the environment information sensor to an external device and to receive a control signal from the external device, and a controller to control operations of the vibrator and the display device based on at least one of the sensed value of the brainwave measuring sensor, the sensed value of the environment sensor, and the control signal.

If at least one of the sensed value of the brainwave measuring sensor and the sensed value of the environment information sensor meets a preset dangerous condition or a control signal to inform the user of a dangerous state is received, the controller may display a warning message through the display device and control the vibrator to generate the vibrations.

The wearable device may further include an output device to output an informing signal to inform of an existence of the user, such that if a preset event occurs, the controller may control the output device to output the informing signal, and the informing signal may include at least one of a light-emitting diode (LED) flickering signal and an informing sound.

The wearable device may further include a sensor to sense a remaining amount of a medium necessary to allow the user to survive, such that if the remaining amount of the medium is lower than a preset threshold value, the controller may display a warning message through the display device and control the vibrator to generate the vibrations.

The wearable device may further include a timer to count a time, such that if the wearable device is activated, the controller may control the timer to start the counting and, if the counted time reaches a preset limit time, display an informing message through the display device and control the vibrator to generate the vibrations.

The wearable device may further include a first body part that the user wears on a head of the user, and a second body part that the user wears on an upper body part of the user, such that the brainwave measuring sensor may be disposed in the first body part, the vibrator may be disposed in at least one of shoulder and neck positions of the second body part, the display device may be disposed in at least one of a plurality of arm positions of the second body part, and the output device may be disposed on a back of the second body part.

The foregoing and/or other features and utilities of the present general inventive concept may also be achieved by providing a status managing method of a wearable device that a user wears on a body, the status managing method including sensing a brainwave and surrounding environment information by using a brainwave measuring sensor and an environment information sensor attached to the wearable device, transmitting the brainwave and the surrounding environment information to an external device, and if at least one of a sensed value of the brainwave measuring sensor and a sensed value of the environment information sensor meets a preset dangerous condition or a control signal to inform the user of a dangerous state is received from the external device, controlling a display device and a vibration motor attached to the wearable device to inform the user of the dangerous state.

The status managing method may further include, if a preset event occurs, outputting an informing signal to inform of a position of the user.

The status managing method may further include sensing a remaining amount of a medium necessary to allow the user to survive, and if the remaining amount of the medium is lower than a preset threshold value, providing a warning message and vibrations.

The status managing method may further include, if the wearable device is activated, starting to count a time, and if the counted time reaches a preset limit time, providing an informing message and vibrations.

The foregoing and/or other features and utilities of the present general inventive concept may also be achieved by providing a managing device including a communicator to receive brainwave information and surrounding environment information sensed by a brainwave measuring sensor and an environment information sensor from a wearable device including the brainwave measuring sensor, the environment information sensor, and a display device, a storage device to store a dangerous condition of a brainwave and a surrounding environment of the user, and a controller to compare the brainwave information and the surrounding environment information with the dangerous condition to determine whether the user is in a dangerous state and, if it is determined that the user is in the dangerous state, generates a control signal to generate a warning message and a vibration signal, and transmits the control signal to the wearable device.

The communicator may communicate with a plurality of wearable devices, such that if a first wearable device of the plurality of wearable devices is in a dangerous state, the controller may inform the other wearable devices of a status of the first wearable device.

The foregoing and/or other features and utilities of the present general inventive concept may also be achieved by providing a status managing method of a managing device, the status managing method including receiving brainwave information and surrounding environment information sensed by a brainwave measuring sensor and an environment information sensor from a wearable device including the brainwave measuring sensor, the environment information sensor, and a display device, determining whether the wearable device is in a dangerous state by comparing the brainwave information and the surrounding environment information with a preset dangerous condition, and if it is determined that the wearable device is in the dangerous state, generating a control signal to generate a warning message and a vibration signal and transmitting the control signal to the wearable device.

The status managing method may further include if it is determined that a first wearable device of a plurality of wearable devices connected to the managing device is in a dangerous state, informing the other wearable devices of a status of the first wearable device.

The foregoing and/or other features and utilities of the present general inventive concept may also be achieved by providing a wearable device that a user wears on a body of the user, the wearable device including a brainwave measuring sensor to sense a brainwave of the user, an environment information sensor to sense surrounding environment information, and a controller to alert the user of danger based on at least one of the sensed brainwave and the sensed surrounding environment information.

The wearable device may further include a display device to display a message corresponding to a type of the danger.

The wearable device may further include a vibrator to generate vibrations at an intensity proportional to a type of the danger.

The wearable device may further include a communicator to transmit sensed values of at least one of the brainwave measuring sensor and the environment information sensor to an external device and to receive a control signal including instructions on how to avoid the danger from the external device, and at least one of a display device and a vibrator to alert the user of the danger.

The controller may control operations of the at least one of the vibrator and the display device based on at least one of the sensed value of the brainwave measuring sensor, the sensed value of the environment sensor, and the control signal.

The communicator may alert other wearable devices of the danger the user is experiencing via wireless transmission.

The wearable device may further include an output device to alert other wearable devices of the danger the user is experiencing via at least one of text and sound.

The wearable device may further include a sensor to sense whether a survival medium utilized by the user is near depletion and to send a signal to the controller to alert the user of the near depletion.

The wearable device may further include a timer to monitor a working time of the user and to send a signal to the controller to alert the user that the working time has exceeded or is about to exceed a predetermined time.

The alert may be with respect to the user's danger.

### BRIEF DESCRIPTION OF THE DRAWINGS

The above and/or other features and utilities of the present general inventive concept will become more apparent and more readily appreciated from the following description of the exemplary embodiments with reference to the accompanying drawings, of which:

FIG. 1 is a block diagram illustrating a structure of a wearable device according to an exemplary embodiment of the present general inventive concept;

FIG. 2 is a view illustrating an appearance structure of a wearable device according to an exemplary embodiment of the present general inventive concept;

FIG. 3 is a view illustrating a warning message provided from a wearable device according to an exemplary embodiment of the present general inventive concept;

FIG. 4 is a view illustrating a structure of a battery provided in a wearable device according to an exemplary embodiment of the present general inventive concept;

FIG. 5 is a view illustrating a cross-section structure of the battery of FIG. 4;

FIG. 6 is a block diagram illustrating a structure of a wearable device according to another exemplary embodiment of the present general inventive concept;

FIG. 7 is a view illustrating a structure of a user status managing system according to an exemplary embodiment of the present general inventive concept;

FIG. 8 is a flowchart illustrating a status managing method performed in a wearable device according to an exemplary embodiment of the present general inventive concept;

FIG. 9 is a flowchart illustrating a status managing method performed in a wearable device according to another exemplary embodiment of the present general inventive concept;

FIG. 10 is a block diagram illustrating a structure of a managing device according to an exemplary embodiment of the present general inventive concept; and

FIG. 11 is a flowchart illustrating a status managing method performed in a managing device according to an exemplary embodiment of the present general inventive concept.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures.

In the following description, the same drawing reference numerals are used for the same elements even in different drawings. The matters defined in the description, such as detailed construction and elements, are provided to assist in a comprehensive understanding of the exemplary embodiments. Thus, it is apparent that the exemplary embodiments can be carried out without those specifically defined matters. Also, well-known functions or constructions are not described in detail since they would obscure the exemplary embodiments with unnecessary detail.

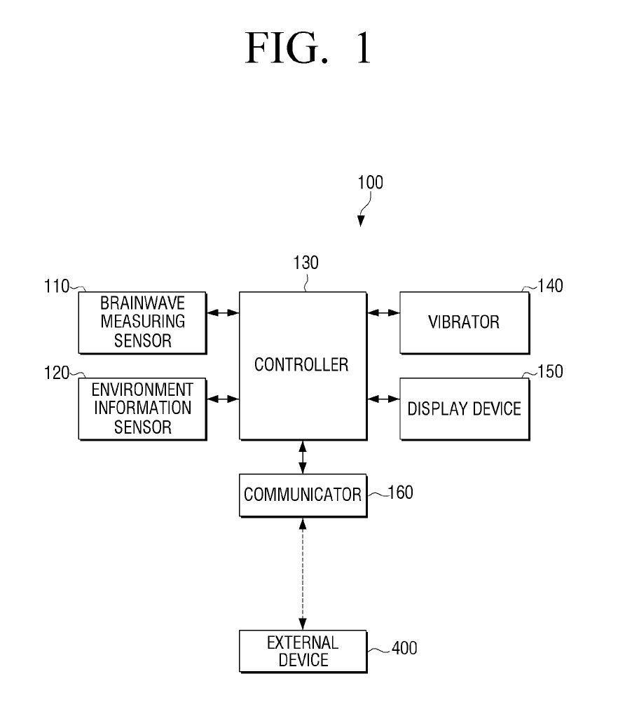

FIG. 1 is a block diagram illustrating a structure of a wearable device 100 according to an exemplary embodiment of the present general inventive concept. The wearable device 100 refers to a device that is formed of a flexible material and that a user may wear. For example, the wearable device 100 may be various types of wearable devices that humans or animals may wear on their bodies, such as clothes, shoes, glasses, hats, accessories, etc., but is not limited thereto. Hereinafter, a wearable device that may include a shape of clothes will be described, but is not limited to this type. Therefore, the wearable device 100 may include various types.

Referring to FIG. 1, the wearable device 100 includes a brainwave measuring sensor 110, an environment information sensor 120, a controller 130, a vibrator 140, a display device 150, and a communicator 160.

The brainwave measuring sensor 110 measures a brainwave signal of a user that wears the wearable device 100. The user may be a human or another living organism, but exemplary embodiments of the present general inventive concept will be directed to the user being a human in the present specification.

The brainwave measuring sensor 110 measures an electrical signal of a brain of a user received from an electrode attached onto a head of the user. In detail, the brainwave measuring sensor 110 detects a current flowing on a scalp surface of the user. A detected brainwave includes a Delta wave, a Theta wave, Alpha wave, a Beta wave, a Gamma wave, etc. The brainwave measuring sensor 110 provides the sensed brainwave signal to the controller 130. The brainwave measuring sensor 110 may extract the brain wave in a non-analgesic method or a non-bonding method by using a silver fiber electrode installed on the head of the user. The brainwave measuring sensor 110 removes a noise signal different from a blink signal from the brainwave signal and outputs the resulting signal. The controller 130 determines a concentration, a laxity, a consciousness or an unconsciousness, etc. of the user based on the resulting signal.

The environment information sensor 120 senses surrounding environment information. The surrounding environment information refers to various types of information through which a surrounding environment of the user may be determined. In detail, the environment information sensor 120 includes at least one or more of various types of sensors such as a temperature sensor, a humidity sensor, an illuminance sensor, an oxygen amount sensor, a poisonous gas amount sensor, a radioactivity sensor, etc. Therefore, the environment information sensor 120 may sense various types of surrounding environment information such as a temperature, humidity, illuminance, an oxygen amount, a poisonous gas amount, a radioactivity amount, etc. A poisonous gas may be various types of harmful gases such as a methane gas, carbon monoxide, etc. The environment information sensor 120 provides the sensed surrounding environment information to the controller 130.

The vibrator 140 is attached to the wearable device 100 to generate vibrations. The vibrator 140 may include various types of vibrators such as a vibration motor, a piezoelectric element, an actuator, etc.

The display device 150 displays various types of messages. The display device 150 may be realized as a liquid crystal display (LCD) panel, a light-emitting diode (LED) array, etc., but is not limited thereto

The communicator 160 communicates with various types of external devices 400. In detail, the communicator 160 transmits sensing values of the brainwave measuring sensor 110 and the environment information sensor 120 to an external device 400 and receives various types of signals from the external device 400. The communicator 160 may perform communications by using a communication method set with respect to the wearable device 100 among various types of communication methods such as Bluetooth, Zigbee, near field communication (NFC), radio frequency (RF) wireless communication standards, etc.

If it is determined that the user wearing the wearable device 100 is in a dangerous state, the controller 130 warns the user of the dangerous state. Whether the user is in the dangerous state may be determined by the controller 130 or by the external device 400. In other words, the controller 130 controls operations of the vibrator 140 and the display device 150 based on the sensing value of the brainwave measuring sensor 110, the sensing value of the environment information sensor 120, a control signal transmitted from the external device 400, etc., but is not limited thereto.

According to an exemplary embodiment of the present general inventive concept, if at least one of the sensing value of the brainwave measuring sensor 110 and the sensing value of the environment information sensor 120 meets a preset danger condition, the controller 130 may control the operations of the vibrator 140 and the display device 150 to warn the user of the dangerous state.

The preset danger condition may be set with respect to each of the brainwave and the environment information. For example, the brainwave is sensed as a Delta wave between 0.1 Hz and 3 Hz in a deep sleep state and as a Theta wave between 4 Hz and 7 Hz in a shallow sleep state. Therefore, a situation in which the Delta wave or the Theta wave is detected may be set to a dangerous condition. In other words, if the Delta wave or the Theta wave is detected, this situation may be recognized that the user falls unconscious. Therefore, the controller 130 determines this situation as a dangerous state.

When environment information is transmitted to the communicator 160, a situation in which a temperature, humidity, an oxygen amount, a poisonous gas amount, a radioactivity level, or the like goes into a dangerous level may be set to the dangerous condition. If at least one of these environment information pieces goes into the dangerous condition, the controller 130 determines this situation as a dangerous state.

If the dangerous state is determined, the controller 130 displays a warning message through the display device 150 or controls the vibrator 140 to generate vibrations. The warning message and the vibrations may be provided simultaneously or separately.

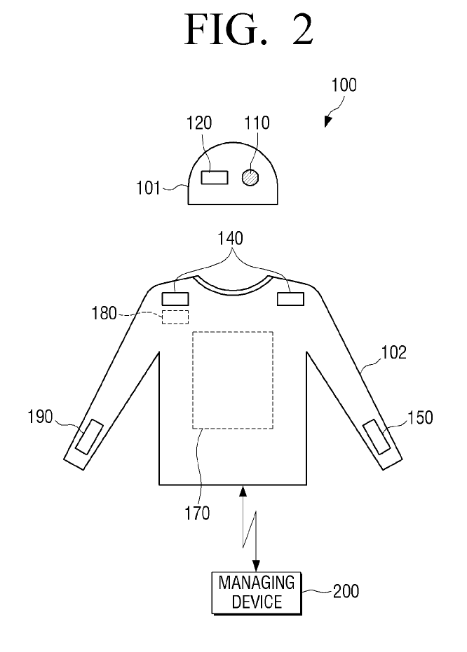

According to another exemplary embodiment of the present general inventive concept, the controller 130 may transmit the sensing values of the brainwave measuring sensor 110 and the environment information sensor 120 to an external managing device 200, as illustrated in FIG. 2. The external managing device 200 may determine whether the user is in the dangerous state, based on the sensing values transmitted from the wearable device 100 and, if it is determined that the user is in the dangerous state, transmit a control signal to inform the user of the dangerous state. If the control signal is received, the controller 130 may control operations of the vibrator 140 and the display device 150 to provide a warning message and vibrations.

According to another exemplary embodiment of the present general inventive concept, the controller 130 may respond to the dangerous state in different ways according to situations by using the sensing values of the brainwave measuring sensor 110 and the environment information sensor 120.

In other words, if it is determined that the surrounding environment information is within a dangerous range, the controller 130 controls the display device 150 to display a warning message.

If it is determined that the brainwave of the user is in an abnormal state, i.e., the user falls unconscious, the controller 130 generates a warning message and vibrations together and informs the external managing device 200 of a dangerous situation. The external managing device 200 informs an emergency center, a hospital, a manager, a terminal device of a surrounding worker, etc., that the user wearing the wearable device 100 is in a dangerous situation. Therefore, even when the user falls unconscious, the user may be rapidly rescued.

According to another exemplary embodiment, the controller 130 may determine whether the user is in the dangerous situation, based on the sensing values of the brainwave measuring sensor 110 and the environment information 120. The controller 130 may transmit the sensing values to the external managing device 200 separately from the determination to enable the external managing device 200 to determine whether the user is in the dangerous situation. If it is determined that the user is in the dangerous situation or a control signal is received from the external managing device 200, the controller 130 may provide a warning message and vibrations. Therefore, although the controller 130 of the wearable device 100 malfunctions, the dangerous situation may be dually checked by the external managing device 200.

FIG. 2 is a view illustrating an appearance structure of a wearable device 100 according to an exemplary embodiment of the present general inventive concept. The wearable device 100 may be realized as an all-in-one suit that covers a user from a body part of the user to a head of the user or as a suit that is divided to separately cover the head and the body part. In FIG. 2, the wearable device 100 is divided into a head part and a body part. Referring to FIG. 2, the wearable device 100 includes a first body part that a user wears on the head and a second body part 102 that the user wears on an upper body. As illustrated in FIG. 2, if the wearable device 100 is divided into the first and second body parts 101 and 102, components of the wearable device 100 are distributed in the first and second body parts 101 and 102. As such, the elements disposed in the first and second body parts 101 and 102 may be connected to one another through a wire or wireless interface.

The first body part 101 may be a hat, a hair band, a hairpin, eyeglasses, a headset, or the like. A brainwave measuring sensor 110 is disposed in the first body part 101. The brainwave measuring sensor 110 may be attached to a head of the user to be connected to the head of the user through wires. An environment information sensor 120 is disposed in the first body part 101 in FIG. 2 but may be disposed in various positions. In detail, a sensor that senses an oxygen amount or a gas amount may be disposed around a respiratory device in the first body part 101, and a temperature sensor or a radioactivity sensor may be disposed in a position closest to the earth surface in the second body part 102.

In FIG. 2, a vibrator 140 may be disposed in at least one of a shoulder position and a neck position in the second body part 102.

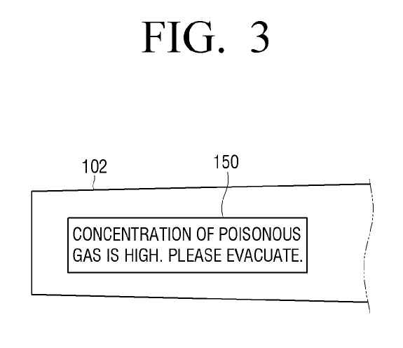

The display device 150 may be disposed in a position that is easily seen by the user. In FIG. 2, the display device 150 is disposed in one arm position in the second body part 102. If plurality of display devices 150 is disposed, the plurality of display devices 150 may be disposed in both arm positions. FIG. 3 is a view illustrating a warning message displayed through the display device 150 according to an exemplary embodiment of the present general inventive concept. Referring to FIG. 3, the display device 150 may be disposed in an arm part to display a warning message, various types of notification messages, graphing images, etc., but is not limited thereto.

Referring to FIG. 2, the wearable device 100 may further include various types of additional elements such as an output device 170, a sensor 180, a timer 190, etc. in addition to the brainwave measuring sensor 110, the environment information sensor 120, the vibrator 140, and the display device 150. The output device 170 outputs a notification signal to notify other users of an existing emergency situation of the user, the sensor 180 senses a remaining amount of a medium necessary the user to survive, and the timer 190 counts time.

Since the output device 170 informs other users of the existence of the user in an emergency situation, the output device 170 is disposed in a position in which the other users may easily recognize the wearable device 100. In the exemplary embodiment of FIG. 2, the output device 170 is disposed on a back part of the second body part 102. The output device 170 may include an LED array. If a preset event occurs, the controller 130 causes flickering lights of an LED of the output device 170 to inform the other users that the user is in a dangerous situation. Here, the preset event may include a situation in which the user falls unconscious, a situation in which an oxygen amount of a respirator that the user wears is depleted to a certain level, a situation in which an error occurs in the wearable device, a situation in which it is difficult to view the user due to lowering of a surrounding illumination intensity, etc. The output device 170 may be disposed on a rear part of the first body part 101 or on the back part of the second body part 102.

The output device 170 may further include a speaker in addition to the LED array. As such, if an event as described above occurs, the controller 130 may output a LED flickering signal and a notification sound simultaneously or alternatingly.

The wearable device 100 may include a storage tank to store a medium such as oxygen or water that the user may use. In this case, the sensor 180 is further included to check a remaining amount of the medium such as oxygen or water. The sensor 180 periodically senses the remaining amount of the medium and provides the sensed remaining amount of the medium to the controller 130. Since the storage tank is typically disposed on the back of the user to allow the user to perform various activities, the sensor 180 may also be disposed on the back of the second body part 102. If the remaining amount of the medium is lower than a preset threshold value, the controller 130 may display a warning message through the display device 150 and control the vibrator 140 to generate vibrations.

If it is determined that oxygen lacks or a concentration of a poisonous gas is higher than or equal to a predetermined reference value, the controller 130 may operate an oxygen generator.

If the timer 190 is installed, the controller 130 may control the timer 190 to start counting a time when the user wears the wearable device 100 or a turn-on button is selected to activate the wearable device 100. The counted time may be displayed through a display means included in the timer 190. The user may check a working time by using the timer 190.

If the counted time reaches a preset limit time, the controller 130 may display a warning message through the display device 150 and control the vibrator 140 to generate vibrations. If a time to measure a working time is installed, and a measured time exceeds an allowed working time, a user is informed of a break time so as to secure safety of the user.

The controller 130 transmits results sensed by the brainwave measuring sensor 110, the environment information sensor 120, the sensor 180, the timer, etc. to a managing device 200. The managing device 200 transmits various control signals to the wearable device 100 based on various types of information transmitted from the wearable device 100.

The first body part 101 may be formed as a helmet shape, the second body part 102 may be formed as a shirt shape, and the first and second body parts 101 and 102 may be formed of a flexible material. In this case, a power supply device formed of a flexible material may be installed in at least one of the first and second body parts 101 and 102.

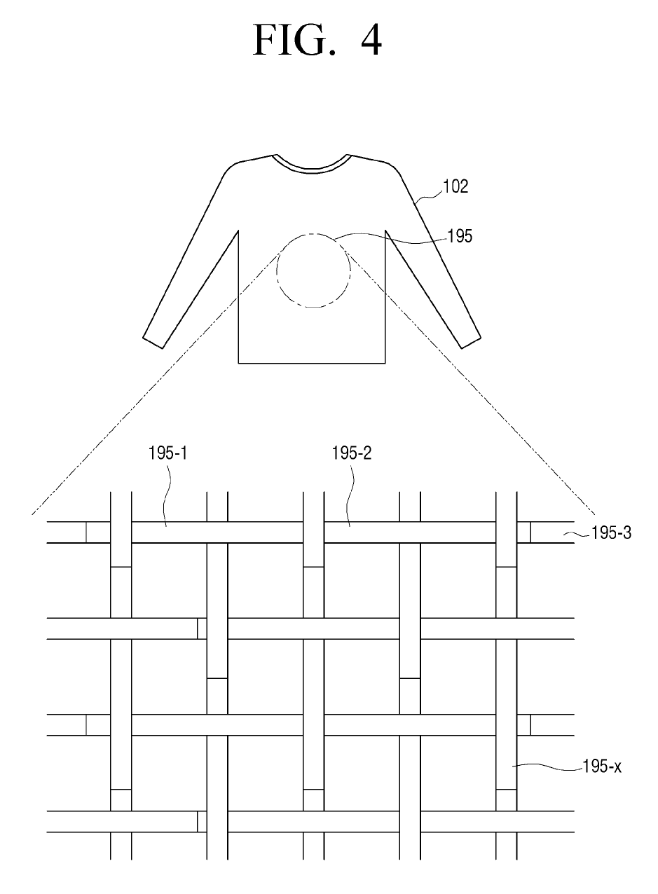

FIG. 4 is a view illustrating a structure of a wearable device including a power supply device 195 formed of a flexible material according to an exemplary embodiment of the present general inventive concept. The power supply device 195 may be a primary battery or a secondary battery. The power supply device 195 may also be formed of the flexible material to correspond appropriately to various characteristics and flexibilities of the wearable device.

In FIG. 4, a plurality of line batteries 195-1, 195-2, 195-3, . . . , and 195-x are connected to one another in series to form one line, and formed lines intersect with one another to form a fabric structure. The power supply device 195 is disposed in a portion of a second body part 102 of the wearable device in FIG. 4, but a whole part of the wearable device may be included as the power supply device 195. In other words, as illustrated in FIG. 4, a plurality of line batteries having flexible characteristics may be connected to one another to form a fabric structure that has a clothing shape and supports elements such as various types of sensors, a controller, etc.

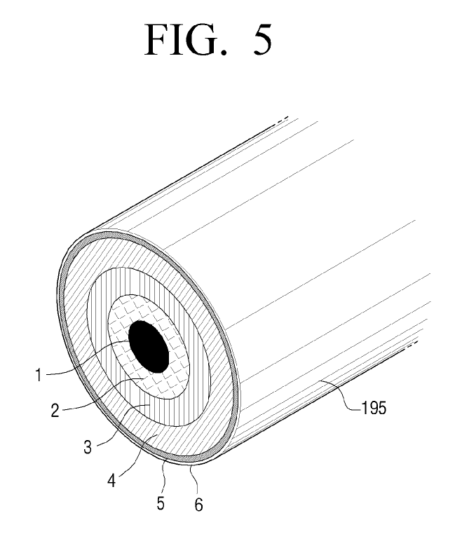

FIG. 5 is a view illustrating an internal structure of one line battery 195 according to an exemplary embodiment of the present general inventive concept. Referring to FIG. 5, the line battery 195 includes an internal current collector 1, an internal electrode 2, an electrolytic part 3, an external electrode 4, an external current collector 5, and a coating 6 that are sequentially disposed from an inside portion of the battery 195 to an outside portion of the battery 195.

The internal current collector 1 may be formed of an alloy such as TiNi having an elastic characteristic, a carbon fiber, other conductive polymers, or the like. A surface of the internal current collector 1 is covered by the internal electrode 2. The internal electrode 2 may be formed of various types of materials according to a characteristic thereof. If the internal electrode 2 is used as a negative electrode, the internal material 2 may be formed of a negative electrode material such as lithium, natrium, or the like. In this case, the external electrode 4 is used as a positive electrode and thus may be formed of a positive electrode material such as sulfur (S), metal sulfide, etc., but is not limited thereto. If the internal electrode 2 is used as a positive electrode, and the external electrode 4 is used as a negative electrode, the internal electrode 2 may be formed of a positive electrode material, and the external electrode 4 may be formed of a negative electrode material. A surface of the internal electrode 2 is covered with the electrolytic part 3. The electrolytic part 3 physically isolates the internal and external electrodes 2 and 4 from each other so as to exchange ions between the internal and external 2 and 4. The electrolytic part 3 may be formed in various forms such as a gel form, a porous form, a solid state form, etc. The external electrode 4 is disposed on an outer side of the electrolytic part 3, and the external current collector 5 is disposed on an outer side of the external electrode 4. The external current collector 5 may be formed of various materials like the internal current collector 5 that has been described above. The coating 6 is formed on an outer side of the external current collector 5. The coating 6 may be formed of a general polymer resin. For example, polyvinyl chloride (PVC), an epoxy resin, or the like may be used. Any material that prevents damage to a thread-shape battery and is freely bent or crooked may be used as the coating 6. The structure of the line battery 195 of FIG. 5 is only an example and thus is not limited thereto.

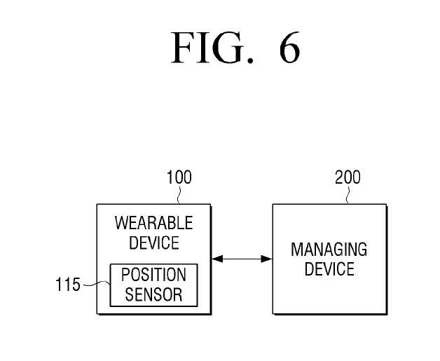

FIG. 6 is a view illustrating a structure of a wearable device 100 according to an exemplary embodiment of the present general inventive concept. Referring to FIG. 6, the wearable device 100 further includes a position sensor 115 in addition to elements illustrated in FIG. 1 or 2.

The position sensor 115 senses a position of a user. In detail, the position sensor 115 may include a geomagnetic sensor and a calorie tracker. The position sensor 115 senses an azimuth by using the geomagnetic sensor and measures the number of steps. The controller 130 combines the azimuth and the number of steps to calculate a position of the wearable device 100. The controller 130 transmits the position of the wearable device 100 to the managing device 200.

If it is determined that the user of the wearable device 100 is in a dangerous situation, the managing device 200 informs various types of external devices 400, such as a terminal device of a surrounding user, an emergency center, etc., of the position of the wearable device 100.

The wearable device 100 is mainly used in a particular environment in which a global positioning system (GPS) chip may not normally used, as in a manhole, a nuclear reactor, etc., but is not limited thereto. The wearable device 100 according to the present exemplary embodiment directly calculates and uses the position of the user of the wearable device 100 based on the azimuth and the number of steps. Therefore, the wearable device 100 may check the position of the user even in an environment in which a GPS chip may not be used.

The managing device 200 may be a portable device of the user of the wearable device 100 such as a portable phone, a tablet PC, laptop PC, etc., but is not limited thereto. Alternatively, the managing device 200 may be one managing device that generally manages a working environment in which a plurality of users work together.

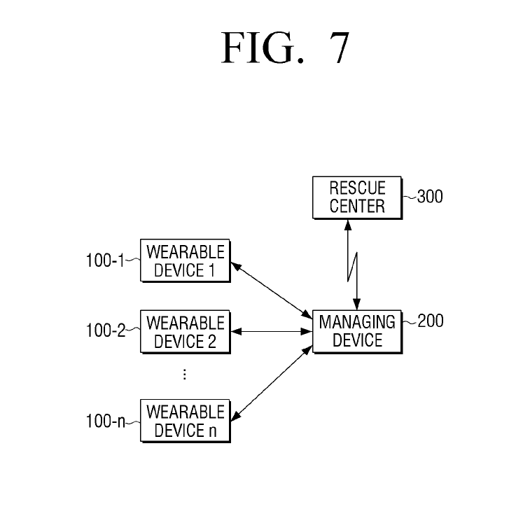

FIG. 7 is a block diagram illustrating a structure of a user status managing system according to an exemplary embodiment of the present general inventive concept. Referring to FIG. 7, the user status managing system includes a plurality of wearable devices 100-1, 100-2 . . . , and 100-n and a managing device 200.

The wearable devices 100-1, 100-2, . . . , and 100-n may include various structures as described above. The wearable devices 100-1, 100-2, . . . , and 100-n respectively brainwaves and surrounding environment information of respective users to the managing device 200.

The managing device 200 compares the brainwaves and the surrounding environment information received from the wearable devices 100-1, 100-2, . . . , and 100-n with a preset dangerous condition to determine whether the users are in dangerous states. Therefore, if it is determined that the users are in dangerous states, the managing device 200 generates a control signal to generate a warning message and a vibration signal and transmits the control signal to the corresponding wearable devices.

If it is determined that the first wearable device 100-1 of the plurality of wearable devices 100-1, 100-2, . . . , and 100-n is in a dangerous state, the managing device 200 transmits the dangerous state of the first wearable device 100-1 to the other wearable devices 100-2, . . . , and 100-n. The managing device 200 transmits an emergency rescue request to a rescue center 300. Moreover, if a second wearable device 100-2 perceives that the first wearable device 100-1 may be in danger, the second wearable device 100-2 may communicate to any other wearable device 100-1, 100-3, 100-4, . . . , and 100-n, or to the managing device 200, of the danger present to the first wearable device 100-1. More specifically, a user of the second wearable device 100-2 may have his/her brainwaves measured by the brainwave measuring sensor 110 such that a particular brainwave level corresponds to anxiety related to perceiving another user in imminent danger. As such, a brainwave level based on perceiving another user in imminent danger would be lower than that of a brainwave level based on perceiving oneself in imminent danger. Accordingly, users of the plurality of wearable devices 100-1, 100-2, . . . , and 100-n can monitor each others' safety.

The managing device 200 synthesizes, accumulates, and stores the surrounding environment information received from the wearable devices 100-1, 100-2, . . . , and 100-n. Therefore, the managing device 200 may define characteristics of working environments. The managing device 200 may pre-register information about the wearable devices 100-1, 100-2, . . . , and 100-n. The managing device 200 may pre-store normal brainwave information and body information of the users of the wearable devices 100-1, 100-2, . . . , and 100-n. The preset dangerous condition may be adaptively set to appropriately correspond to characteristics of the users.

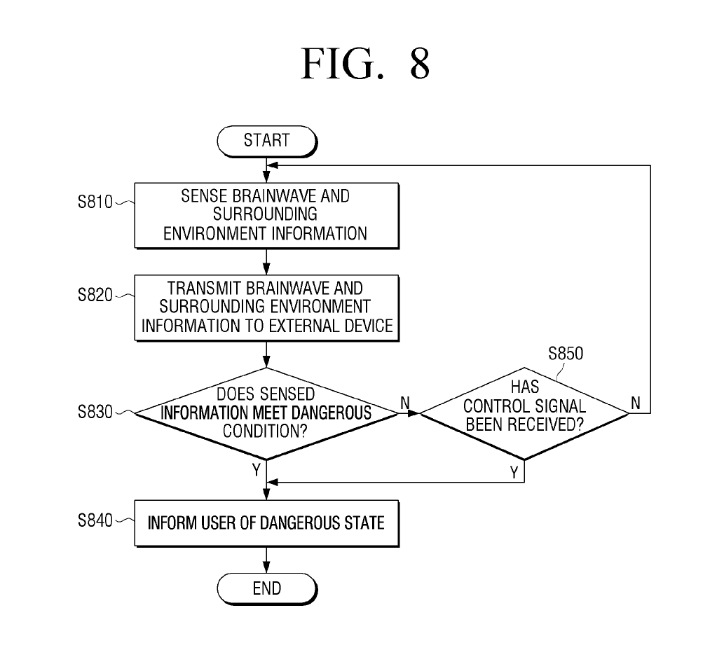

FIG. 8 is a flowchart illustrating a status managing method performed in a wearable device according to an exemplary embodiment of the present general inventive concept.

Referring to FIG. 8, in operation S810, the wearable device senses a brainwave and surrounding environment information. In operation S820, the wearable device transmits the brainwave and the surrounding environment information to an external device.

If the wearable device determines that the sensed information meets a preset dangerous condition in operation S830 or determines that a control signal has been received from the external device in operation S850, the wearable device informs a user that the user is in a dangerous state, by using a display device or a vibrator in operation S840. A method of informing the user of the dangerous state or a requirement to inform the user of the dangerous state may be variously realized as described above.

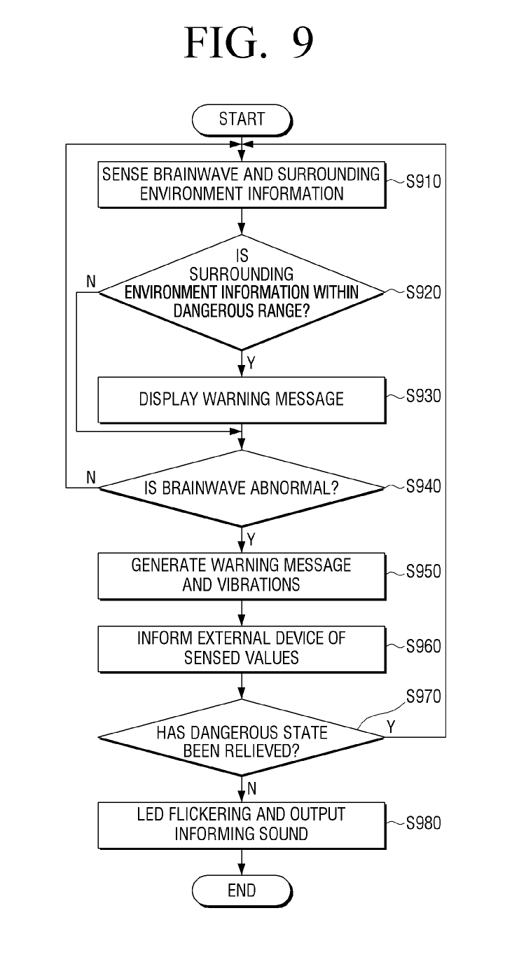

FIG. 9 is a flowchart illustrating a status managing method according to another exemplary embodiment of the present general inventive concept. Referring to FIG. 9, in operation S910, a wearable device senses a brainwave and surrounding environment information. The wearable device first analyzes the sensed result to determine whether a user is in a dangerous state. In operation S920, the wearable device determines whether the surrounding environment information is within a dangerous range. If it is determined in operation S920 that the surrounding environment information is within the dangerous state, the wearable device outputs a warning message in operation S930.

If the wearable device senses that the brainwave is in an abnormal state in operation S940, the wearable device generates a warning message and vibrations in operation S950. Accordingly, if a speaker is included, the wearable device may provide a notification sound together.

The vibrations may be provided even when the surrounding environment information is within the dangerous range. However, intensities of the vibrations may be different according to various different situations. For example, when the surrounding environment information is within the dangerous range, weak vibrations may be provided to call the user's attention to the dangerous range. When the brainwave of the user is in the abnormal state, i.e., the user loses consciousness, strong vibrations may be provided.

In operation S960, the wearable device informs an external device of these sensed values. The external device informs other communicable devices of a status of the user of the wearable device to perform an emergency rescue.

In operation S970, the wearable device determines whether the dangerous state has relieved. If it is determined in operation S970 that the dangerous state lasts, the wearable device makes flickering lights of an LED of an output device attached to the wearable device or outputs a notification sound through a speaker to inform surrounding users of an existence of the user of the wearable device in operation S980. The output device may include a flexible LED.

As described above, if the present wearable device is used, a dangerous situation is rapidly checked and prevented. In an emergency situation, like when a user loses consciousness, steps are taken to recover the user's consciousness, and a rescue request is transmitted to an external device. Also, a rescue of the user may be easily performed by using an LED flickering signal and a notification sound to inform other users of an existence of the user of the wearable device.

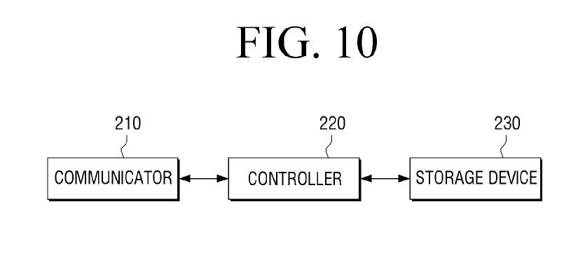

FIG. 10 is a block diagram illustrating a structure of the managing device 200 according to an exemplary embodiment of the present general inventive concept. Referring to FIG. 10, the managing device 200 includes a communicator 210, a controller 220, and a storage device 230.

The communicator 210 receives brainwave information and surrounding environment information from a wearable device including various types of elements such as a brainwave measuring sensor, an environment information sensor, a vibrator, a display device, etc. The communicator 210 may include at least one of various types of chips such as a WiFi chip, a Bluetooth chip, a near field communication (NFC) chip, a wireless communication chip, etc. If the communicator 210 uses the WiFi chip or the Bluetooth chip, the communicator 210 may transmit and receive various types of connection information such as a subsystem identification (SSID), a session key, etc. with the wearable device to transmit and receive various types of information, a command, etc., with the wearable device after a communication connection. The wireless communication chip performs a communication according to various communication standards such as Institute of Electrical and Electronics Engineers (IEEE), Zigbee, 3rd Generation (3G), 3rd Generation Partnership Project (3GPP), Long Term Evolution (LTE), etc.

The storage device 230 stores programs such as Operating System (O/S) software, various types of applications, etc. to drive the managing device 200, various types of data input or set when executing the programs, and various types of data such as dangerous condition information, etc. In detail, the storage device 230 stores a program to manage a status of a user.

The controller 220 executes the program stored in the storage device 230 to analyze a bio-signal and surrounding environment information received from the wearable device in order to manage a status of a worker. In detail, the controller 220 compares the brainwave information and the surrounding environment information received through the communicator 210 with a dangerous condition to determine whether the user is in a dangerous state. If it is determined that the user is in the dangerous state, the controller 220 generates a control signal to generate a warning message and a vibration signal and transmits the control signal to the wearable device through the communicator 210.

The controller 220 may accumulate and manage information and statuses of users sensed by respective wearable devices. The managing device 200 may provide information that the managing device 200 manages, to a web server. Therefore, the managing device 200 may provide information about respective workers to a third person or a manager. The manager may check information about a worker in a working environment online at any time and at any location.

The managing device 200 illustrated in FIG. 10 may be a user terminal device such as a portable phone or a tablet PC or may be realized as all-in-one managing device that manages a plurality of wearable devices.

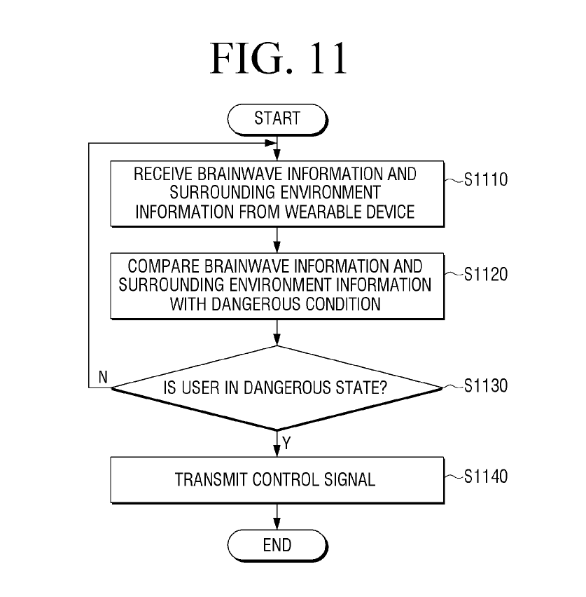

FIG. 11 is a flowchart illustrating a status managing method of a managing device according to an exemplary embodiment of the present general inventive concept. Referring to FIG. 11, in operation S1110, the managing device receives brainwave information and surrounding environment information from a wearable device. In operation S1120, the managing device compares the received information with a dangerous condition. If it is determined in operation S1130 that a user is in a dangerous state, the managing device transmits a control signal in operation S1140.

Alternatively, the status managing method may further include, if it is determined that a first wearable device of a plurality of wearable devices in a dangerous state is in a dangerous state, informing the wearable devices of a status of the first wearable device.

The above-described wearable device may further include various types of sensors such as a sensor to sense an electrocardiogram (ECG), an electromyogram (EMG) sensor, etc. Various types of elements, such as various types of sensors, a controller, a display device, a communicator, etc. attached to a wearable device, may be realized as shapes that are attached to or detached from the wearable device. Alternatively, the wearable device may be realized as a bag-shaped module that a user may carry.

According to various exemplary embodiments of the present general inventive concept as described above, a wearable device that a user wears senses a status and surrounding environment information of the user to immediately inform the user of whether the user is in a dangerous state. Therefore, a dangerous situation of the user is prevented, and the user may rapidly react and respond to the dangerous situation, although the dangerous situation occurs.

The present general inventive concept can also be embodied as computer-readable codes on a computer-readable medium. The computer-readable medium can include a computer-readable recording medium and a computer-readable transmission medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network coupled computer systems so that the computer-readable code is stored and executed in a distributed fashion. The computer-readable transmission medium can transmit carrier waves or signals (e.g., wired or wireless data transmission through the Internet). Also, functional programs, codes, and code segments to accomplish the present general inventive concept can be easily construed by programmers skilled in the art to which the present general inventive concept pertains.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A wearable device that a user wears on a body of the user, the wearable device comprising:
a brainwave measuring sensor to measure a brainwave of the user;
an environment information sensor to sense surrounding environment information;
a vibrator to generate vibrations;
a display device to display a message;
a communicator to transmit sensed values of the brainwave measuring sensor and the environment information sensor to an external device and to receive a control signal from the external device; and
a controller to control operations of the vibrator and the display device based on at least one of the sensed value of the brainwave measuring sensor, the sensed value of the environment sensor, and the control signal.

2. The wearable device of claim 1, wherein if at least one of the sensed value of the brainwave measuring sensor and the sensed value of the environment information sensor meets a preset dangerous condition or a control signal to inform the user of a dangerous state is received, the controller displays a warning message through the display device and controls the vibrator to generate the vibrations.

3. The wearable device of claim 2, further comprising:
an output device to output an informing signal to inform of an existence of the user,
wherein if a preset event occurs, the controller controls the output device to output the informing signal, and the informing signal comprises at least one of a light-emitting diode (LED) flickering signal and an informing sound.

4. The wearable device of claim 3, further comprising:
a sensor to sense a remaining amount of a medium necessary to allow the user to survive,
wherein if the remaining amount of the medium is lower than a preset threshold value, the controller displays a warning message through the display device and controls the vibrator to generate the vibrations.

5. The wearable device of claim 4, further comprising:
a timer to count a time,
wherein if the wearable device is activated, the controller controls the timer to start the counting and, if the counted time reaches a preset limit time, displays an informing message through the display device and controls the vibrator to generate the vibrations.

6. The wearable device of claim 5, further comprising:
a first body part that the user wears on a head of the user; and
a second body part that the user wears on an upper body part of the user,
wherein the brainwave measuring sensor is disposed in the first body part, the vibrator is disposed in at least one of shoulder and neck positions of the second body part, the display device is disposed in at least one of a plurality of arm positions of the second body part, and the output device is disposed on a back of the second body part.

7. A status managing method of a wearable device that a user wears on a body, the status managing method comprising:
sensing a brainwave and surrounding environment information by using a brainwave measuring sensor and an environment information sensor attached to the wearable device;
transmitting the brainwave and the surrounding environment information to an external device; and
if at least one of a sensed value of the brainwave measuring sensor and a sensed value of the environment information sensor meets a preset dangerous condition or a control signal to inform the user of a dangerous state is received from the external device, controlling a display device and a vibration motor attached to the wearable device to inform the user of the dangerous state.

8. The status managing method of claim 7, further comprising:
if a preset event occurs, outputting an informing signal to inform of a position of the user.

9. The status managing method of claim 8, further comprising:
sensing a remaining amount of a medium necessary to allow the user to survive; and
if the remaining amount of the medium is lower than a preset threshold value, providing a warning message and vibrations.

10. The status managing method of claim 9, further comprising:
if the wearable device is activated, starting to count a time; and
if the counted time reaches a preset limit time, providing an informing message and vibrations.

11. A managing device comprising:
a communicator to receive brainwave information and surrounding environment information sensed by a brainwave measuring sensor and an environment information sensor from a wearable device comprising the brainwave measuring sensor, the environment information sensor, and a display device;
a storage device to store a dangerous condition of a brainwave and a surrounding environment of the user; and
a controller to compare the brainwave information and the surrounding environment information with the dangerous condition to determine whether the user is in a dangerous state and, if it is determined that the user is in the dangerous state, generates a control signal to generate a warning message and a vibration signal, and transmits the control signal to the wearable device.

12. The managing device of claim 11, wherein the communicator communicates with a plurality of wearable devices,
wherein if a first wearable device of the plurality of wearable devices is in a dangerous state, the controller informs the other wearable devices of a status of the first wearable device.

13. A status managing method of a managing device, the status managing method comprising:
receiving brainwave information and surrounding environment information sensed by a brainwave measuring sensor and an environment information sensor from a wearable device comprising the brainwave measuring sensor, the environment information sensor, and a display device;
determining whether the wearable device is in a dangerous state by comparing the brainwave information and the surrounding environment information with a preset dangerous condition; and
if it is determined that the wearable device is in the dangerous state, generating a control signal to generate a warning message and a vibration signal and transmitting the control signal to the wearable device.

14. The status managing method of claim 13, further comprising:
if it is determined that a first wearable device of a plurality of wearable devices connected to the managing device is in a dangerous state, informing the other wearable devices of a status of the first wearable device.

15. A wearable device that a user wears on a body of the user, the wearable device comprising:
a brainwave measuring sensor to sense a brainwave of the user;
an environment information sensor to sense surrounding environment information;
a communicator to transmit sensed values of at least one of the brainwave measuring sensor and the environment information sensor to an external device and to receive a control signal; and
a controller to alert the user of danger based on at least one of the sensed brainwave and the sensed surrounding environment information.

16. The wearable device of claim 15, further comprising:
a display device to display a message corresponding to a type of the danger.

17. The wearable device of claim 15, further comprising:
a vibrator to generate vibrations at an intensity proportional to a type of the danger.

18. The wearable device of claim 15, wherein the control signal includes instructions on how to avoid the danger from the external device, and
further comprising at least one of a display device and a vibrator to alert the user of the danger.

19. The wearable device of claim 15, wherein the controller controls operations of the at least one of the vibrator and the display device based on at least one of the sensed value of the brainwave measuring sensor, the sensed value of the environment sensor, and the control signal.

20. The wearable device of claim 15, wherein the alert is with respect to the user's danger.

21. The wearable device of claim 1, wherein the external device is a mobile phone.
